(19) United States
(12) Patent Application Publication
(10) Pub. No.: US 2020/0016343 A1
(43) Pub. Date: Jan. 16, 2020

(54) System for Precise Injection of Nano-Formulations

(71) Applicants: Alex HAREL, Savion (IL); NANOMED TARGETING SYSTEMS INC, Oklahoma City, OK (US)

(72) Inventor: Alex HAREL, Savion (IL)

(73) Assignee: Nanomed Targeting Systems Inc, Oklahoma City, OK (US)

(21) Appl. No.: 16/490,592

(22) PCT Filed: Mar. 5, 2018
(86) PCT No.: PCT/IL2018/050243

§ 371 Date: Sep. 2, 2019

(30) Foreign Application Priority Data
Mar. 14, 2017 (IL) 251140

(51) Int. Cl. A61M 5/42; A61M 5/46; A61M 5/31

(52) U.S. Cl. CPC A61M 5/427; A61M 5/46; A61M 5/31; A61M 2205/3306; A61M 2205/50; A61M 2005/3125; A61M 2209/084; A61M 2205/3313; A61M 2205/587

(57) ABSTRACT

A system for the precise and accurate injection of therapeutic substances in a small specific site in the patient's body that includes a syringe, a miniature video camera, lighting means, control buttons, a power source, and a camera-display connecting means. The miniature video camera is positioned on the syringe and the lens of the camera is positioned on the front end of the syringe and is directed towards the needle/needle array tip area. The lighting means are positioned on the front end of the syringe and are designed to illuminate the needle tip area. The control buttons are designed to control the camera and the lighting means. The camera-display connecting means is designed to connect the camera to a display, which is designed to show the area as photographed by the camera.

### TECHNICAL FIELD

The present invention refers to a system for the precise injection of therapeutic substances such as nano-formulations.

### BACKGROUND ART

It is often necessary to perform an accurate and precise injection in a specific site on the body of a patient. For example, a proposed treatment for postoperative atrial fibrillation involves an injection of a nano-formulation to the ganglion plexus of the patient. To achieve such accuracy, it is necessary to visualize and enlarge the injection area during the injection, without compromising visibility, and to support the syringe so that the user can deliver the injection to the exact, desired site. The present patent application describes a system for precise and accurate injections.

### LIST OF DRAWINGS

The intention of the drawings attached to the application is not to limit the scope of the invention and its application. The drawings are intended only to illustrate the invention and they constitute only one of its many possible implementations.

FIG. 1 is a schematic depiction of the system (1) that includes a syringe (2), a needle (23), a miniature video camera (3), lighting means (4) and control buttons (5).

FIG. 2 is a schematic depiction of the nano-formulation aggregation testing system (400) and the system (1) wherein the syringe (2) is assembled on the supporting stand (8).

FIG. 3 depicts the syringe holder (200).

FIG. 4 depicts schematically the depth control mechanism (500).

### THE INVENTION

The main objective of the present invention is to provide a system for the precise and accurate injection of therapeutic substances in a small specific site in the patient's body. The system (1) includes a syringe (2), a miniature video camera (3), lighting means (4), and control buttons (5). The general structure of the syringe (2) may be that of a standard syringe.

The miniature video camera (3) is positioned on the syringe (2) wherein the lens (31) is positioned on the front end (21) of the syringe (2) facing the needle/needle array tip area (22). The lighting means (4) may be miniature light bulbs or small light-emitting diodes (LED). The lighting means (4) are positioned on the front end (21) of the syringe (2) and are directed towards the needle tip area (22) which they are designed to illuminate.

The system (1) also includes an inner battery or a connection to an outer power source (hereinafter “power source” (6)) through a transformer for example. The power source (6) is designed to supply electrical power to the camera and the lighting means. The control buttons (5) are designed to control the miniature video camera and the lighting means. For example, they may control the zoom feature and the contrast or brightness of the camera, the light intensity of the lighting means, and the on/off switch on the camera and the lighting means.

The system (1) also includes a camera-display connecting means (71) that connects the miniature video camera (3) to a display (72), which shows the area as photographed by the camera. This camera-display connecting means (71) can be a cable connector or transmitter for wireless connection, such as WiFi, RF or Bluetooth.

The system (1) may also include a supporting stand (8) to which the syringe (2) is assembled so as to enable the user to bring the needle/needle array (23) of the syringe (2) to the desired injection site. The supporting stand (8) may include a base (82) and controlling arms (81) to which the syringe (2) is assembled. The connection of the base (82) to the controlling arms (81) may be axially and flexible.

The system (1) enables the user to view the needle tip or the needle array tips and the targeted injection area, on the display, in one single frame and in an enlarged view, thus enabling the user to guide the needle tip to the desired site. The supportive stand (8) enables the user to guide the needle tip in a relaxed manner and without the need to constantly hold the syringe, an action that involves muscle tension and requires mental concentration.

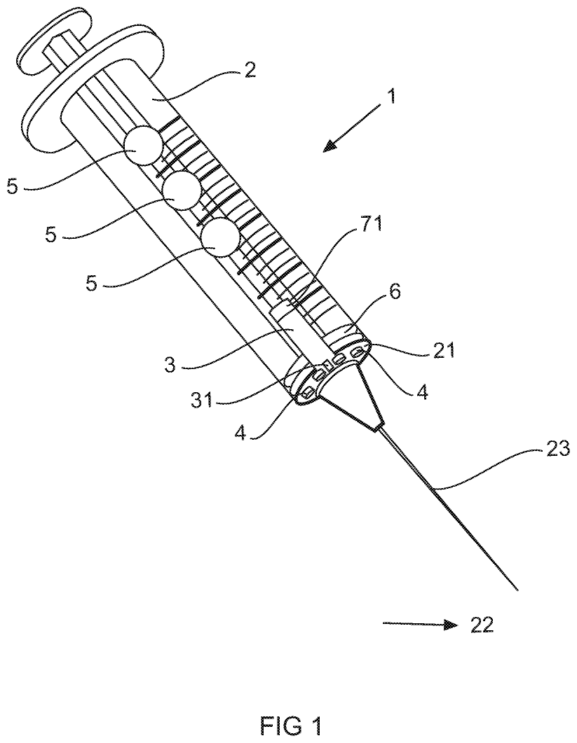

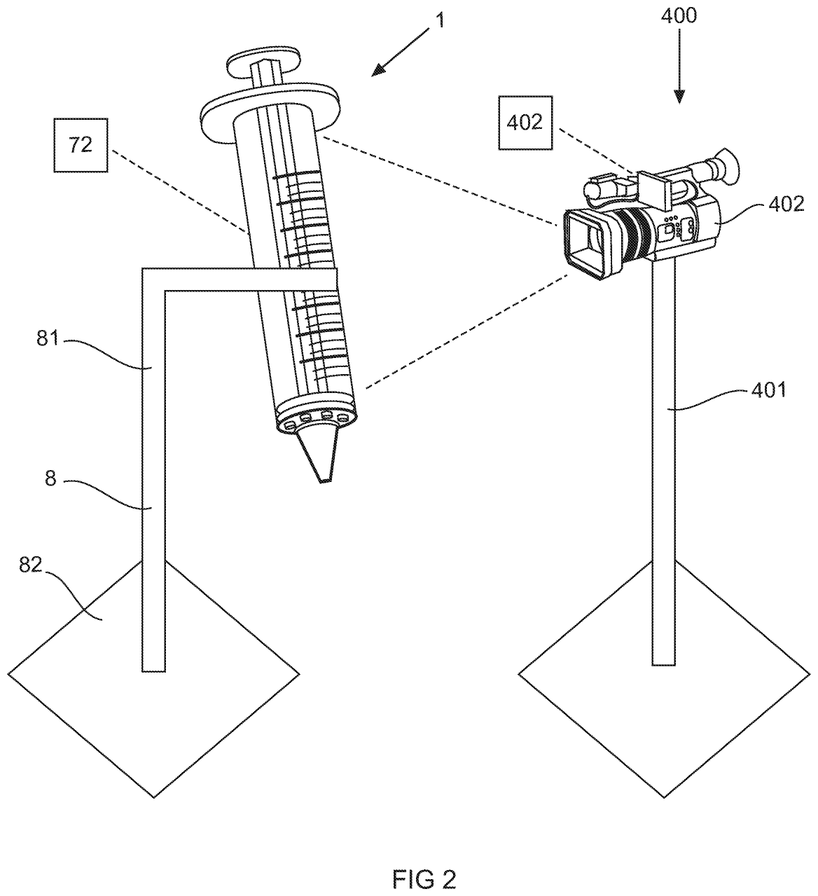

FIG. 1 is a schematic depiction of the system (1) that includes the syringe (2), the needle/needles array (23), the miniature video camera (3), the lighting means (4) and the control buttons (5). FIG. 2 is a schematic depiction of the system (1) wherein the syringe (2) is assembled on the supporting stand (8).

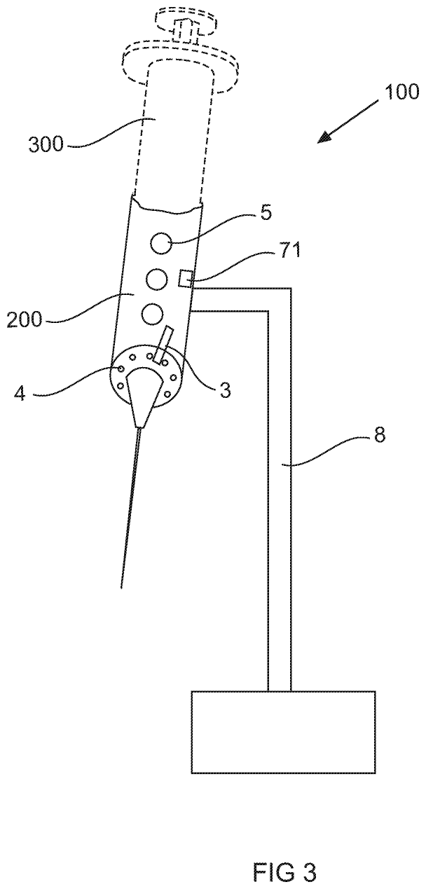

The second embodiment of the present invention refers to the system (100) that includes a syringe holder (200) that is equipped with a miniature video camera (3), lighting means (4), control buttons (5), a power source (6), and the camera-display connecting means (71). The syringe holder (200) is designed to receive and hold a standard syringe (300), as depicted for example in FIG. 3, thus enabling the user to achieve the same goal as with the system (1) but using a standard syringe (300).

The systems (1) and (100) are designed to be used when a precise injection of therapeutic substances is required. When such substances are nano-formulations, the systems (1) and (100) may also include a nano-formulation aggregation testing system (400), which enables the user to determine whether the nano-formulation in the syringe (2) or (300) is ready for use. The nano-formulation aggregation testing system (400) includes a stand (401), a video camera (402), a display (403) and a processor (404). The video camera (402) is directed towards the syringe (2) or (300), which in this case is made of a transparent material and contains the nano-formulation. The processor (404) then analyzes the video film using an image-processing technique that determines whether the nano-formulation in the syringe (2) or (300) is ready for use. This processing technique may check, among other parameters, the color uniformity of the nano-formulation solution, whether the solution includes lumps, and whether there is any aggregation. FIG. 2 illustrates the nano-formulation aggregation testing system (400).

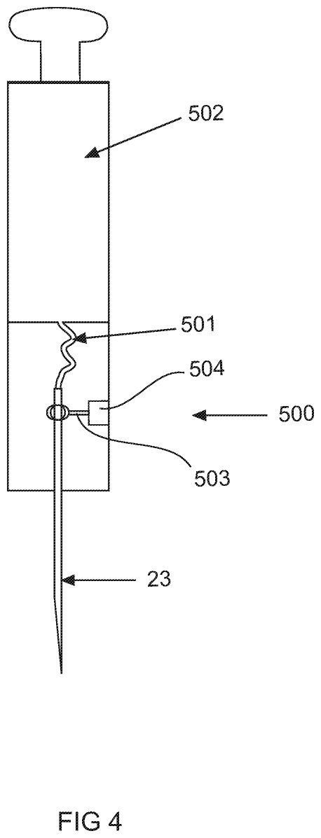

The system (1) may further include a depth control mechanism (500) that is designed to control the depth that the needle (23) will penetrate the tissue. The therapeutic substances flow from the syringe container (502) to the needle (23) through a narrow pipe (501). The needle (23) is connected to a moving arm (503) that can be move down and up by an activator (504), which may be operated by a spring, air pressure, electricity or any other method. The user controls the activator to which the needle (23) is attached and by that the user control the depth in which the needle may be penetrates the tissue. Also in this embodiment the system may include one needle or several. FIG. 4 depicts schematically the depth control mechanism (500).

What is claimed is:

1. A system for the precise and accurate injection of therapeutic substances into a small, specific site in the patient's body, that comprises a syringe, a miniature video camera, lighting means, control buttons, a power source, and a camera-display connecting means;
wherein the miniature video camera is positioned on the syringe and wherein the lens of said miniature video camera is positioned on the front end of the syringe and is directed towards the needle/needle array tip area;
wherein the lighting means are positioned on the front end of the syringe and are designed to illuminate the needle/needle array tip area;
wherein the control buttons are designed to control the miniature video camera and the lighting means;
wherein the camera-display connecting means is designed to connect the miniature video camera to a display; wherein the display is designed to show an area as photographed by the camera;
wherein the system enables a user to view the needle tip and the targeted injection area, on the display, in one single frame and in an enlarged view, thus enabling the user to guide the needle tip to the desired site.

2. A system for the precise and accurate injection of therapeutic substances into a small, specific site in the patient's body, comprising: a syringe holder that is designed to receive and hold a standard syringe, a miniature video camera, lighting means, control buttons, a power source, and a camera-display connecting means;
wherein the miniature video camera is positioned on the holder, and wherein lens of said miniature video camera is positioned on the front end of the syringe holder and is directed towards the needle/needle array tip area;
wherein the lighting means are positioned on the front end of the syringe holder and are designed to illuminate the needle/needle array tip area;
wherein the control buttons are designed to control the miniature video camera and the lighting means;
wherein the camera-display connecting means is designed to connect the miniature video camera to the display; wherein the display is designed to show an area as photographed by the camera;

3. The system for the precise and accurate injection of therapeutic substances in a small specific site in the patient's body according to claim 1 that further includes a supporting stand; wherein said syringe is assembled on the supportive stand thus enabling said user to bring said needle of the syringe to said desired injection site; whereby the supportive stand enables the user to guide the needle tip in a relaxed manner and without having to constantly hold the syringe.

4. The system for the precise and accurate injection of therapeutic substances in a small specific site in the patient's body according to claim 2 that further includes a supporting stand; wherein said syringe holder is assembled on the supportive stand thus enabling said user to bring said needle/needle array of said standard syringe to said desired injection site; whereby the supportive stand enables the user to guide the needle tip in a relaxed manner and without having to constantly hold the syringe.

5. The system for the precise and accurate injection of therapeutic substances in a small specific site in the patient's body according to claim 1 that further includes a nano-formulation aggregation testing system, which is designed to enable the user to determine whether the nano-formulation in the syringe is ready for use; wherein the nano-formulation aggregation testing system includes a stand, a video camera, a display, and a processor; wherein the video camera is directed towards the syringe that contains the nano-formulation; the processor analyzes the a video film filmed by the camera using an image-processing technique to determine whether the nano-formulation in the syringe is ready for use; wherein the processing technique may check the color uniformity of the nano-formulation solution, whether the solution contains lumps, and whether there is any aggregation.

6. The system for the precise and accurate injection of therapeutic substances in a small specific site in the patient's body according to claim 2 that further includes a nano-formulation aggregation testing system, which is designed to enable the user to determine whether the nano-formulation in the syringe is ready for use; wherein the nano-formulation aggregation testing system includes a stand, a video camera, a display, and a processor; wherein the video camera is directed towards the syringe that contains the nano-formulation; the processor analyzes the a video film filmed by the camera using an image-processing technique to determine whether the nano-formulation in the syringe is ready for use; wherein the processing technique may check the color uniformity of the nano-formulation solution, whether the solution contains lumps, and whether there is any aggregation.

7. The system for the precise and accurate injection of therapeutic substances in a small specific site in the patient's body according to claim 1 that further includes a depth control mechanism that is designed to control the depth that said needle will penetrate a tissue; wherein said therapeutic substances flow from a syringe container to said needle through a narrow pipe; wherein said needle is connected to a moving arm that can be move down and up by an activator; whereby enabling a user to control the activator to which said needle is attached and by that enables the user to control the depth in which said needle penetrates the tissue.

8. The system for the precise and accurate injection of therapeutic substances in a small specific site in the patient's body according to claim 2 that further includes a depth control mechanism that is designed to control the depth that said needle will penetrate a tissue; wherein said therapeutic substances flow from a syringe container to said needle through a narrow pipe; wherein said needle is connected to a moving arm that can be move down and up by an activator; whereby enabling a user to control the activator to which said needle is attached and by that enables the user to control the depth in which said needle penetrates the tissue.
